(19) United States
(12) Patent Application Publication
(10) Pub. No.: US 2026/0041797 A1
(43) Pub. Date: Feb. 12, 2026

(54) RADIOPAQUE CROSSLINKED ACRYLAMIDE COPOLYMERS AND USES THEREOF

(71) Applicant: Boston Scientific Scimed, Inc., Maple Grove, MN (US)

(72) Inventor: Yen-Hao Hsu, Shrewsbury, MA (US)

(73) Assignee: Boston Scientific Scimed, Inc., Maple Grove, MN (US)

(21) Appl. No.: 19/288,214

(22) Filed: Aug. 1, 2025

Related U.S. Application Data
(60) Provisional application No. 63/680,379, filed on Aug. 7, 2024.

(51) Int. Cl. A61K 49/04; A61M 5/178

(52) U.S. Cl. CPC A61K 49/0442; A61K 49/0457; A61K 49/0476; A61M 5/178; A61K 2123/00

(57) ABSTRACT

In some aspects, the present disclosure pertains to radiopaque crosslinked acrylamide copolymers that comprise (a) a residue of an iodinated monofunctional unsaturated monomer that comprises (i) a single acrylamido group or a single methacrylate group and (ii) one or more iodinated aromatic groups linked to the acrylamido group or the methacrylate group through a linkage comprising a C₂-C₈ alkyl group, (b) a residue of a monofunctional acrylamide monomer that does not contain an iodinated aromatic group, and (c) a residue of a multifunctional acrylamide monomer that does not contain an iodinated aromatic group. Other aspects of the present disclosure pertain to methods of making such radiopaque crosslinked acrylamide copolymers, to particulate compositions containing such radiopaque crosslinked acrylamide copolymers, and to methods of treatment using such particulate compositions.

### CROSS-REFERENCE TO RELATED APPLICATIONS

This application claims the benefit of U.S. Provisional Patent Application Ser. No. 63/680,379 filed on Aug. 7, 2024, the disclosure of which is incorporated herein by reference.

### FIELD

The present disclosure relates to radiopaque crosslinked acrylamide copolymers, particulate compositions comprising radiopaque crosslinked acrylamide copolymers, and methods of making and using radiopaque crosslinked acrylamide copolymers and particulate compositions. The radiopaque crosslinked acrylamide copolymers and particulate compositions of the present disclosure are useful, for example, in various medical procedures.

### BACKGROUND

Polyacrylamide is a widely used hydrogel in the biomedical and biotechnology industries. Polyacrylamide hydrogel is currently used as the sole material component in the product the Bulkamid™. This product has been in medical use for a number of years as a bulking agent in cosmetic surgery and ophthalmic surgery and more recently has been used to treat stress urinary incontinence (SUI) in women. For this latter clinical case, the material remains permanently implanted to allow for relief of symptoms.

### SUMMARY

There is a need in the biomedical arts for radiopaque polyacrylamide materials, and for methods of making and using radiopaque polyacrylamide materials.

In some aspects, the present disclosure pertains to radiopaque crosslinked acrylamide copolymers that comprise (a) a residue of an iodinated monofunctional unsaturated monomer that comprises (i) a single acrylamido group,

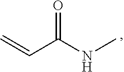

or a single methacrylate group,

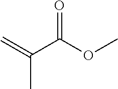

and (ii) one or more iodinated aromatic groups linked to the acrylamido group or the methacrylate group through a linkage comprising a C₂-C₈ alkyl group, (b) a residue of a monofunctional acrylamide monomer that does not contain an iodinated aromatic group, and (c) a residue of a multifunctional acrylamide monomer that does not contain an iodinated aromatic group.

In some aspects, the present disclosure pertains to a radiopaque crosslinked acrylamide copolymer comprising (a) a residue of an iodinated monofunctional unsaturated monomer that comprises (i) a single acrylamido group,

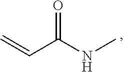

or a single methacrylate group,

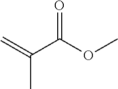

and (ii) one or more iodinated aromatic groups, (b) a residue of a monofunctional acrylamide monomer that does not contain an iodinated aromatic group, and (c) a residue of a multifunctional acrylamide monomer that does not contain an iodinated aromatic group, wherein the one or more iodinated aromatic groups in the radiopaque crosslinked acrylamide copolymer are spaced from a polymer chain into which the residue of the iodinated monofunctional unsaturated monomer is incorporated through a linkage that comprises an alkyl chain that ranges from two to eight carbon atoms in length.

In some embodiments, which can be used in conjunction with the above aspects, the linkage further comprises an amide group or an ether group.

In some embodiments, which can be used in conjunction with the above aspects and embodiments, the iodinated monofunctional unsaturated monomer is selected from an iodinated (C₂-C₈ alkyl)methacrylate monomer and an iodinated N—(C₂-C₈ alkyl)acrylamide monomer, and the one or more iodinated aromatic groups are linked to one or more carbons of the C₂-C₈ alkyl group. In some of these embodiments, the one or more iodinated aromatic groups may be (a) directly linked to the one or more carbons of the C₂-C₈ alkyl group, (b) linked to the one or more carbons of the C₂-C₈ alkyl group through an ether group, and/or (c) linked to the one or more carbons of the C₂-C₈ alkyl group through an amide group.

In some embodiments, which can be used in conjunction with the above aspects and embodiments, the iodinated aromatic groups are iodinated phenyl groups. In some of these embodiments, the iodinated phenyl groups are substituted with one, two, three, four or five iodine atoms. In some of these embodiments, the iodinated phenyl groups are further substituted with one, two, three or four hydrophilic groups. In some of these embodiments, the hydrophilic groups are selected from hydroxyl groups and C₁-C₆-hydroxyalkyl groups. In some of these embodiments, the C₁-C₆-hydroxyalkyl groups are directly linked to the phenyl group, the C₁-C₆-hydroxyalkyl groups are linked to the phenyl group through an ether group and/or the C₁-C₆-hydroxyalkyl groups are linked to the phenyl group through an amide group.

In some embodiments, which can be used in conjunction with the above aspects and embodiments, the monofunctional acrylamide monomer that does not contain an iodinated aromatic group is selected from acrylamide and an N—C₁-C₆-alkylacrylamide.

In some embodiments, which can be used in conjunction with the above aspects and embodiments, multifunctional acrylamide monomer that does not contain an iodinated aromatic group is selected from N,N′-methylenebisacrylamide, N,N′-1,2-ethylenebisacrylamide, N,N′-1,3-trimethylenebisacrylamide, N,N′-1,4-tetramethylenebisacrylamide, N,N′-1,5-pentamethylenebisacrylamide, N,N′-1,7-pentamethylenebisacrylamide, N,N′-1,6-hexamethylenebisacrylamide, N,N′-1,8-octamethylenebisacrylamide, and N,N′-1,12-dodecamethylenebisacrylamide.

In some embodiments, which can be used in conjunction with the above aspects and embodiments, the radiopaque crosslinked acrylamide copolymer further comprises a residue of a multifunctional acrylamide monomer that contains one or more iodinated aromatic groups. In some of these embodiments, the multifunctional acrylamide monomer that contains one or more iodinated aromatic groups is selected from 1,3-bisacrylamido-2,4,6-triiodobenzene, 1,3-bisacrylamido-5-iodobenzene, and 1,3-bisacrylamido-2,4,5,6-tetraiodobenzene.

In some embodiments, which can be used in conjunction with the above aspects and embodiments, the radiopaque crosslinked acrylamide copolymer is a hydrogel.

In some embodiments, which can be used in conjunction with the above aspects and embodiments, the radiopaque crosslinked acrylamide copolymer has a radiopacity that is greater than 100 Hounsfield units (HU).

In other aspects, the present disclosure pertains to a radiopaque particulate composition comprising radiopaque particles that comprise a radiopaque crosslinked acrylamide copolymer in accordance with any of the above aspects and embodiments.

In some embodiments, the radiopaque particulate composition further comprises one or more additional agents selected from one or more of therapeutic agents, imaging agents, colorants, tonicity adjusting agents, suspension agents, wetting agents, and pH adjusting agents.

In some embodiments, which can be used in conjunction with the above aspects and embodiments, the radiopaque particles have an average size ranging from 50 to 950 microns.

In some embodiments, which can be used in conjunction with the above aspects and embodiments, the radiopaque particulate composition is an injectable composition.

In some embodiments, which can be used in conjunction with the above aspects and embodiments, the radiopaque particulate composition is provided in a container in sterile dry form.

In some embodiments, which can be used in conjunction with the above aspects and embodiments, the radiopaque particulate composition is provided in a container in a suspension. In some of these embodiments, the container is selected from a syringe, vial, ampoule or catheter component.

In other aspects, the present disclosure pertains to a medical procedure comprising administering to a subject a radiopaque particulate composition in accordance with any of the above aspects and embodiments.

In some embodiments, the medical procedure is selected from a tissue bulking procedure, a tissue spacing procedure, a blood vessel occlusion procedure, and a fiducial marking procedure. In some of these embodiments, the particles are viewed under x-ray imaging during and/or after administration.

In other aspects, the present disclosure pertains to a method of making a radiopaque crosslinked acrylamide copolymer comprising photopolymerizing an aqueous solution that comprises (a) an iodinated monofunctional unsaturated monomer that comprises (i) a single acrylamido group,

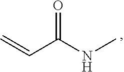

or a single methacrylate group,

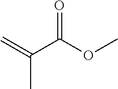

and (ii) one or more iodinated aromatic groups linked to the acrylamido group or the methacrylate group through a linkage comprising a C₂-C₈ alkyl group, (b) a monofunctional acrylamide monomer that does not contain an iodinated aromatic group, (c) a multifunctional acrylamide monomer that does not contain an iodinated aromatic group, and (d) one or more polymerization initiators.

One advantage of the present disclosure is that polyacrylamide materials are provided that are visible under x-ray imaging. This feature allows for a physician to confirm placement location during and after implantation.

The above and other aspects, embodiments, features and benefits of the present disclosure will be readily apparent from the following detailed description.

### BRIEF DESCRIPTION OF THE DRAWINGS

FIG. 1 schematically illustrates methods of forming radiopaque crosslinked acrylamide polymers in accordance with two embodiments of the present disclosure.

FIG. 2 illustrates a delivery device, in accordance with another embodiment of the present disclosure.

FIGS. 3A and 3B schematically illustrate the injection of a radiopaque crosslinked acrylamide polymer composition into a bladder neck of a patient, in accordance with an embodiment of the present disclosure.

### DETAILED DESCRIPTION

In various aspects, radiopaque crosslinked acrylamide polymers are provided herein that comprise a covalently incorporated radiopaque atoms.

In some embodiments, radiopaque crosslinked acrylamide copolymers are provided that comprise (a) a residue of a monofunctional acrylamide monomer that does not contain one or more radiopaque atoms, (b) a residue of a multifunctional acrylamide monomer that does not contain one or more radiopaque atoms, (c) a residue of a radiopaque monofunctional unsaturated monomer that contains one or more radiopaque atoms and (d) optionally, a residue of a multifunctional acrylamide monomer containing one or more radiopaque atoms.

Radiopaque atoms may be selected, for example, from Br, I, Bi, Ba, Gd, Ta, Zn and Au, among others, thereby making the polymer more absorptive of x-rays and thus more visible under x-ray imaging techniques such as x-ray fluoroscopy and computerized tomography (CT) scanning.

In some embodiments, radiopaque crosslinked acrylamide copolymers are provided that comprise (a) a residue of a monofunctional acrylamide monomer that does not contain one or more radiopaque atoms, (b) a residue of a multifunctional acrylamide monomer that does not contain one or more radiopaque atoms, (c) a residue of an iodinated monofunctional unsaturated monomer that comprises (i) a single acrylamido group,

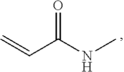

or a single methacrylate group,

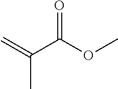

and (ii) one or more iodinated aromatic groups and (d) optionally, a residue of a multifunctional acrylamide monomer containing one or more radiopaque atoms.

In some embodiments, the iodinated aromatic groups in the radiopaque crosslinked acrylamide copolymers are spaced from the polymer chain into which the residue of the iodinated monofunctional unsaturated monomer is incorporated through an alkyl chain that is two, three, four, five, six, or more carbon atoms in length. Varying the length of the alkyl chain is useful, for example for tuning the overall hydrophilicity/hydrophobicity of the radiopaque crosslinked acrylamide copolymers.

Examples of iodinated aromatic groups include iodine-substituted monocyclic aromatic groups and iodine-substituted multicyclic aromatic groups, such as iodinated phenyl groups and iodinated naphthyl groups, among others. The iodinated aromatic groups may be substituted with one, two, three, four, five, six or more iodine atoms. In various embodiments, the aromatic groups may be further substituted with one or more hydrophilic groups, for example, one, two, three, four, five, six or more hydrophilic groups. The hydrophilic groups may be hydroxyl-containing groups, which may be selected, for example, from hydroxyl groups and hydroxyalkyl groups (e.g., C₁-C₄-hydroxyalkyl groups containing one, two, three, four, five or six carbon atoms and one, two, three, four, five, six or more hydroxyl groups, such as C₁-C₄-monohydroxyalkyl groups, C₁-C₄-dihydroxyalkyl groups, C₁-C₄-trihydroxyalkyl groups, C₁-C₄-tetrahydroxyalkyl groups, C₁-C₄-pentahydroxyalkyl groups, C₁-C₄-hexahydroxyalkyl groups, etc.), which hydroxyalkyl groups may be directly linked to the aromatic group or may be linked to the aromatic group through an ether or an amide group.

Particular examples of iodinated phenyl groups may be selected from a mono-hydroxy-monoiodo-phenyl group, a mono-hydroxy-diiodo-phenyl group, a mono-hydroxy-triiodo-phenyl group, a mono-hydroxy-tetraiodo-phenyl group, a di-hydroxy-monoiodo-phenyl group, a di-hydroxy-diiodo-phenyl group, a di-hydroxy-triiodo-phenyl group, a tri-hydroxy-monoiodo-phenyl group, and a tri-hydroxy-diiodo-phenyl group, a mono-C₁-C₄-hydroxyalkyl-monoiodo-phenyl group, a mono-C₁-C₄-hydroxyalkyl-diiodo-phenyl group, a mono-C₁-C₄-hydroxyalkyl-triiodo-phenyl group, a mono-C₁-C₄-hydroxyalkyl-tetraiodo-phenyl group, a di-C₁-C₄-hydroxyalkyl-monoiodo-phenyl group, a di-C₁-C₄-hydroxyalkyl-diiodo-phenyl group, a di-C₁-C₄-hydroxyalkyl-triiodo-phenyl group, a tri-C₁-C₄-hydroxyalkyl-monoiodo-phenyl group, and a tri-C₁-C₄-hydroxyalkyl-diiodo-phenyl group, among others.

In some embodiments, the iodinated monofunctional unsaturated monomer comprises (i) a single acrylamido group or a single methacrylate group and one or more iodinated aromatic groups linked to the acrylamido group or the methacrylate group through a linkage comprising a C₂-C₈ alkyl group, which linkage may optionally further comprise, for example, an amide group or an ether group. C₂-C₈ alkyl groups for use herein include branched and unbranched C₂-C₈ alkyl groups, for example, ethyl, n-propyl, isopropyl, n-butyl, isobutyl, tert-butyl, n-pentyl, isopentyl, neopentyl, tert-pentyl, n-hexyl, isohexyl, neohexyl, n-heptyl, isoheptyl, neoheptyl, n-octyl, isooctyl, and neooctyl groups, among others. Varying the number of carbon atoms in the selected C₂-C₈ alkyl group is useful, for example, for tuning the overall hydrophilicity/hydrophobicity of the iodinated monofunctional unsaturated monomer, and thus the overall hydrophilicity/hydrophobicity of the radiopaque crosslinked acrylamide copolymer into which the iodinated monofunctional unsaturated monomer is incorporated.

Particular examples of iodinated monofunctional unsaturated monomers that contain one or more radiopaque atoms include iodinated (C₂-C₈ alkyl)methacrylate monomers and iodinated N—(C₂-C₈ alkyl)acrylamide monomers where one or more carbons of the C₂-C₈ alkyl groups of the iodinated (C₂-C₈ alkyl)methacrylate monomers and iodinated N—(C₂-C₈ alkyl)acrylamide monomers are substituted with one or iodinated aromatic groups, examples of which are described above, among others, which may be directly linked to the to the C₂-C₈ alkyl group or may be linked to the C₂-C₈ alkyl group through a linkage comprising an ether group or an amide group.

In some embodiments, the C₂-C₈ alkyl groups of the iodinated (C₂-C₈ alkyl)methacrylate monomers and iodinated N—(C₂-C₈ alkyl)acrylamide monomers may be substituted with one or more iodinated phenyl groups, examples of which are described above, among others, which may be directly linked to the C₂-C₈ alkyl group or may be linked to the C₂-C₈ alkyl group through a linkage comprising an ether group (e.g., where an iodinated phenoxy group is linked to the C₂-C₈ alkyl group) or through a linkage comprising an amide group (e.g. where an iodinated phenylaminocarbonyl group or an iodinated phenylcarbonylamino group is linked to the C₂-C₈ alkyl group). In some embodiments, the iodinated phenyl group is substituted with one, two, three, four, five, six or more iodine atoms and, optionally, one, two, three or four hydrophilic groups, which may be selected, for example, from hydroxyl groups and hydroxyalkyl groups (e.g., C₁-C₆-hydroxyalkyl groups containing one, two, three, four, five or six carbon atoms), which hydroxyalkyl groups may be directly linked to the phenyl group or may be linked to the phenyl group through an ether or an amide group.

Specific examples of iodinated N—(C₂-C₈ alkyl)acrylamide monomers include N-(2-(iodinated-aromatic-substituted)ethyl)acrylamide (yielding a radiopaque crosslinked acrylamide copolymer where the iodinated aromatic group is spaced from the polymer chain into which the acrylamide monomer is incorporated through an alkyl chain that is two carbon atoms in length), N-(3-(iodinated-aromatic-substituted)propyl)acrylamide (yielding a radiopaque crosslinked acrylamide copolymer where the iodinated aromatic group is spaced from the polymer chain into which the acrylamide monomer is incorporated through an alkyl chain that is three carbon atoms in length), N-(4-(iodinated-aromatic-substituted)butyl)acrylamide(yielding a radiopaque crosslinked acrylamide copolymer where the iodinated aromatic group is spaced from the polymer chain into which the acrylamide monomer is incorporated through an alkyl chain that is four carbon atoms in length), N-(5-(iodinated-aromatic-substituted)pentyl)acrylamide(yielding a radiopaque crosslinked acrylamide copolymer where the iodinated aromatic group is spaced from the polymer chain into which the acrylamide monomer is incorporated through an alkyl chain that is five carbon atoms in length), N-(6-(iodinated-aromatic-substituted)hexyl)acrylamide(yielding a radiopaque crosslinked acrylamide copolymer where the iodinated aromatic group is spaced from the polymer chain into which the acrylamide monomer is incorporated through an alkyl chain that is six carbon atoms in length), N-(7-(iodinated-aromatic-substituted)heptyl)acrylamide (yielding a radiopaque crosslinked acrylamide copolymer where the iodinated aromatic group is spaced from the polymer chain into which the acrylamide monomer is incorporated through an alkyl chain that is seven carbon atoms in length), and N-(8-(iodinated-aromatic-substituted)octyl)acrylamide (yielding a radiopaque crosslinked acrylamide copolymer where the iodinated aromatic group is spaced from the polymer chain into which the acrylamide monomer is incorporated through an alkyl chain that is eight carbon atoms in length). One particular monomer, which used in the example set forth below, is an N-(4-(iodophenyl)butyl) acrylamide, more particularly, an N-(4-(triiodophenyl)butyl) acrylamide,

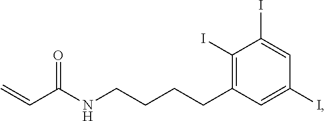 [SMILES: C=CC(=O)NCCCCc1cc(I)cc(I)c1I]

which yields a radiopaque crosslinked acrylamide copolymer where the triiodophenyl is spaced from the polymer chain into which the acrylamide monomer is incorporated through an alkyl chain that is four carbon atoms in length.

Specific examples of iodinated (C₂-C₈ alkyl)methacrylate monomers include 2-(iodinated-aromatic-substituted)ethyl)methacrylate (yielding a radiopaque crosslinked acrylamide copolymer where the iodinated aromatic group is spaced from the polymer chain into which the methacrylate monomer is incorporated through an alkyl chain that is two carbon atoms in length), 3-(iodinated-aromatic-substituted)propyl)methacrylate (yielding a radiopaque crosslinked acrylamide copolymer where the iodinated aromatic group is spaced from the polymer chain into which the methacrylate monomer is incorporated through an alkyl chain that is three carbon atoms in length), 4-(iodinated-aromatic-substituted)butyl)methacrylate(yielding a radiopaque crosslinked acrylamide copolymer where the iodinated aromatic group is spaced from the polymer chain into which the methacrylate monomer is incorporated through an alkyl chain that is four carbon atoms in length), 5-(iodinated-aromatic-substituted)pentyl)methacrylate(yielding a radiopaque crosslinked acrylamide copolymer where the iodinated aromatic group is spaced from the polymer chain into which the methacrylate monomer is incorporated through an alkyl chain that is five carbon atoms in length), 6-(iodinated-aromatic-substituted)hexyl)methacrylate(yielding a radiopaque crosslinked acrylamide copolymer where the iodinated aromatic group is spaced from the polymer chain into which the methacrylate monomer is incorporated through an alkyl chain that is six carbon atoms in length), 7-(iodinated-aromatic-substituted)heptyl)methacrylate (yielding a radiopaque crosslinked acrylamide copolymer where the iodinated aromatic group is spaced from the polymer chain into which the methacrylate monomer is incorporated through an alkyl chain that is seven carbon atoms in length), 8-(iodinated-aromatic-substituted)octyl)methacrylate (yielding a radiopaque crosslinked acrylamide copolymer where the iodinated aromatic group is spaced from the polymer chain into which the methacrylate monomer is incorporated through an alkyl chain that is eight carbon atoms in length).

Iodinated N—(C₂-C₈ alkyl)acrylamide monomers can also be formed by reacting with acrylic acid with a suitable iodinated amine compound in an amide coupling reaction.

Examples of iodinated amine compounds include those that comprise one or more monocyclic or multicyclic aromatic structures, such as a benzene or naphthalene structure, substituted with (a) a substituent comprising a C₂-C₈ primary amine group (referred to herein as a C₂-C₈ amino group), (b) one or more iodine groups (e.g., one, two, three, four, five, six or more iodine atoms) and (c) optionally, one or more hydrophilic groups, such as hydroxy-containing groups (e.g., one, two, three, four, five, six or more hydroxy-containing groups). Hydroxy-containing groups may be independently selected from one or more hydroxy groups and/or one or more C₁-C₄-hydroxyalkyl groups (e.g., C₁-C₄-monohydroxyalkyl groups, C₁-C₄-dihydroxyalkyl groups, C₁-C₄-trihydroxyalkyl groups, C₁-C₄-tetrahydroxyalkyl groups, etc.), which C₁-C₄-hydroxyalkyl groups may be linked to the monocyclic or multicyclic aromatic structures directly or through any suitable linking moiety, which may be selected, for example, from an amide group and an ether group, among others.

Particular examples of iodinated amine compounds include C₂-C₈-amino-substituted-iodo-aromatic compounds include C₂-C₈-amino-substituted-mono-iodo-benzene compounds, C₂-C₈-amino-substituted-di-iodo-benzene compounds, C₂-C₈-amino-substituted-tri-iodo-benzene compounds, C₂-C₈-amino-substituted-tetra-iodo-benzene compounds, C₂-C₈-amino-substituted-penta-iodo-benzene compounds, C₂-C₈-amino-substituted-mono-iodo-naphthylene compounds, C₂-C₈-amino-substituted-di-iodo-naphthylene compounds, C₂-C₈-amino-substituted-tri-iodo-naphthylene compounds, C₂-C₈-amino-substituted-tetra-iodo-naphthylene compounds, C₂-C₈-amino-substituted-penta-iodo-naphthylene compounds, C₂-C₈-amino-substituted-hexa-iodo-naphthylene compounds and C₂-C₈-amino-substituted-hepta-iodo-naphthylene compounds, among others.

Examples of C₂-C₈ primary amine groups include aminoethyl, aminopropyl, aminobutyl, aminopentyl, aminohexyl groups, aminoheptyl groups and aminooctyl groups, such as 2-aminoethyl, 3-amino-n-propyl, 4-amino-n-butyl, 5-amino-n-pentyl, 6-amino-n-hexyl groups, 7-amino-n-heptyl groups and 8-amino-n-octyl groups.

More specific examples of iodinated amine compounds 3-(2,3,5-triiodophenyl)propan-1-amine,

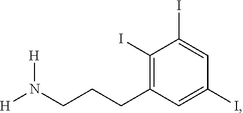 [SMILES: NCCCc1cc(I)cc(I)c1I]

4-(2-iodophenyl)butan-1-amine,

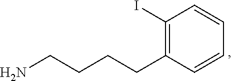 [SMILES: NCCCCc1ccccc1I]

7-(4-iodophenyl)hexan-1-amine,

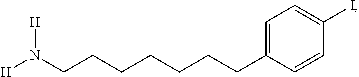 [SMILES: NCCCCCCCc1ccc(I)cc1]

5-(2-iodophenyl)pentan-1-amine,

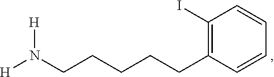 [SMILES: NCCCCCc1ccccc1I]

and 6-(4-iodophenyl)hexan-1-amine

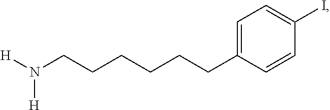 [SMILES: NCCCCCCc1ccc(I)cc1]

among many others.

Iodinated (C₂-C₈-alkyl)methacrylate monomers can be formed, for example, by reacting 2-hydroxymethyl methacrylate with a suitable iodinated alcohol compound (e.g., commercially available contrast agent such as iodixanol, iohexol, and ioversol, among others) in coupling reaction using a suitable coupling agent (e.g., a carbodiimide coupling agent such as N,N′-dicyclohexylcarbodiimide (DCC), N,N′-dicyclohexylcarbodiimide (DCC), etc.).

Examples of monofunctional acrylamide monomers that do not contain one or more radiopaque atoms include acrylamide, N—C₁-C₆-alkylacrylamides, such as N-methylacrylamide, N-ethylacrylamide, N-n-propylacrylamide, N-isopropylacrylamide, etc., and C₁-C₆-alkylacrylamides, such as methacrylamide, ethacrylamide, n-propylacrylamide, isopropacrylamide, etc., among others.

Examples of multifunctional acrylamide monomers that do not contain one or more radiopaque atoms include, for example, N,N′-methylenebisacrylamide, N,N′-1,2-ethylenebisacrylamide, N,N′-1,3-trimethylenebisacrylamide, N,N′-1,4-tetramethylenebisacrylamide, N,N′-1,5-pentamethylenebisacrylamide, N,N′-1,7-pentamethylenebisacrylamide, N,N′-1,6-hexamethylenebisacrylamide, N,N′-1,8-octamethylenebisacrylamide, and N,N′-1,12-dodecamethylenebisacrylamide, among others, each of which can provide varying degrees of hydrophobicity.

Examples of optional multifunctional acrylamide monomers that contain containing one or more radiopaque atoms include 1,3-bisacrylamido-2,4,6-triiodobenzene,

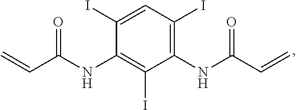 [SMILES: C=CC(=O)Nc1c(I)cc(I)c(NC(=O)C=C)c1I]

1,3-bisacrylamido-5-iodobenzene, and 1,3-bisacrylamido-2,4,5,6-tetraiodobenzene, among others.

In various embodiments, the radiopaque crosslinked acrylamide copolymer is a hydrogel. As used herein, a “hydrogel” is a crosslinked polymer that absorbs water but does not dissolve when placed in water.

Radiopaque crosslinked acrylamide copolymers in accordance with the present disclosure may be made, for example, by photopolymerization, e.g., using visible or ultraviolet light, of an aqueous solution containing a combination of comonomers as set forth above in the presence of one or more suitable initiators such as ammonium persulfate, riboflavin, and N,N,N⁰,N⁰-tetramethylene diamine (TEMED), and/or lithium phenyl-2,4,6-trimethylbenzoyl phosphate (LAP).

Porosity of the resulting radiopaque crosslinked acrylamide copolymer may be varied, for example, by varying the ratio of mono-functional monomers to multi-functional comonomers and/or by varying the total concentration of comonomers in the solution.

Radiopacity of the resulting radiopaque crosslinked acrylamide copolymer may be tuned, for example, by varying a ratio of the radiopaque monofunctional unsaturated monomer containing one or more radiopaque atoms to the monofunctional acrylamide monomer that does not contain one or more radiopaque atoms. Exemplary mol:mol ratios range from 0.5:99.5 to 30:70.

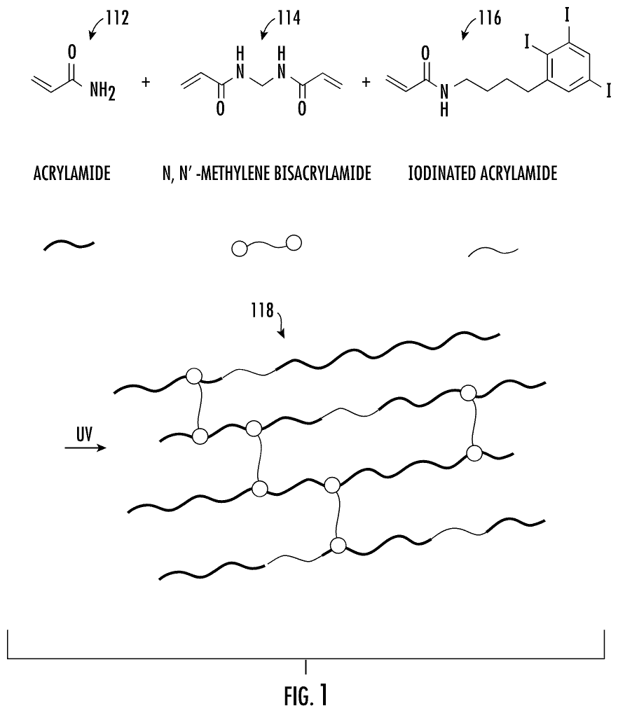

With reference now to FIG. 1, in one embodiment, (i) acrylamide (112), (ii) N,N′-methylenebisacrylamide (114), and (iii) an iodinated N—(C₂-C₈ alkyl)acrylamide monomer, specifically, an N-(4-(triiodophenyl)butyl) acrylamide,

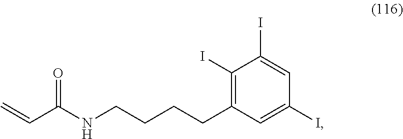 [SMILES: C=CC(=O)NCCCCc1cc(I)cc(I)c1I]

are crosslinked with ultraviolet light in an aqueous solution to form a radiopaque crosslinked acrylamide copolymer hydrogel (118).

The radiopaque crosslinked acrylamide copolymer of the present disclosure may be in any desired form, including a slab, a cylinder, a coating, and a particle (e.g., by emulsion polymerization or bulk polymerization/granulation). In some embodiments, the resulting radiopaque crosslinked acrylamide copolymer may be dried and then granulated into particles of suitable size. Granulating may be by any suitable process, for instance by homogenization, grinding (including cryogrinding), crushing, milling, pounding, or the like. Sieving or other known techniques can be used to classify and fractionate the particles.

In various embodiments, the radiopaque crosslinked acrylamide copolymers in accordance with the present disclosure have a radiopacity that is greater than 100 Hounsfield units (HU), beneficially ranging anywhere from 100 HU to 250 HU to 500 HU to 750 HU to 1000 HU or more (in other words, ranging between any two of the preceding numerical values).

Radiopaque crosslinked acrylamide copolymer particles formed using the above and other techniques may varying widely in size, for example, typically having an average size ranging from 1 to 1000 microns, more typically from 50 to 950 microns.

In various embodiments, compositions are provided which contain a radiopaque crosslinked acrylamide copolymer as described herein as well as one or more additional agents. Such additional agents include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, anti-cancer drugs, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists, among others.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including ⁹⁹mTc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr and ¹⁷⁷Lu, among others, and (f) further radiocontrast agents (beyond the covalently incorporated radiopaque atoms of the hydrogel) such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, polyethylene glycol, polyvinyl alcohol, block copolymers, etc.), among others, and pH adjusting agents including various buffer solutes.

In some embodiments, radiopaque particulate compositions are provided which contain radiopaque crosslinked acrylamide copolymer particles as described herein (also referred to as radiopaque particles) as well as one or more optional additional agents such as those described above (e.g., therapeutic agents, imaging agents, colorants, tonicity adjusting agents, suspension agents, wetting agents, pH adjusting agents, etc.). In particular embodiments, the radiopaque particulate compositions are injectable compositions.

Such radiopaque particulate compositions may be stored and transported in a sterile dry form. The dry composition may be shipped, for example, in a syringe, catheter, vial, ampoule, or other container, and it may be mixed with a suitable liquid carrier (e.g. sterile water for injection. physiological saline, phosphate buffer, a solution containing an imaging contrast agent, etc.) prior to administration. In this way the concentration of the radiopaque particles (and the one or more optional additional agents) in the administered composition may be varied at will, depending on the specific application at hand, as desired by the health care practitioner in charge of the procedure. One or more containers of liquid carrier may also be supplied and shipped, along with the dry radiopaque particles, in the form of a kit.

Such radiopaque particulate compositions may also be stored and transported in form of a sterile suspension that contains water in addition to the radiopaque particles and the one or more optional additional agents. The suspension may be stored, for example, in a syringe, catheter, vial, ampoule, or other container. The suspension may also be mixed with a suitable liquid carrier (e.g. sterile water for injection, physiological saline, phosphate buffer, a solution containing contrast agent, etc.) prior to administration, allowing the concentration of administered radiopaque particles (as well as other optional additional agents) in the suspension to be reduced prior to injection, if so desired by the health care practitioner in charge of the procedure. One or more containers of liquid carrier may also be supplied to form a kit.

In various embodiments, kits are provided that include one or more delivery devices for delivering the radiopaque particulate compositions to a subject.

The radiopaque particulate compositions described herein are visible under x-ray imaging techniques such as x-ray fluoroscopy and computerized tomography (CT) scanning, among others. In various embodiments, the radiopaque particulate compositions have a radiopacity that is greater than 100 Hounsfield units (HU), beneficially ranging anywhere from 100 HU to 250 HU to 500 HU to 750 HU to 1000 HU or more.

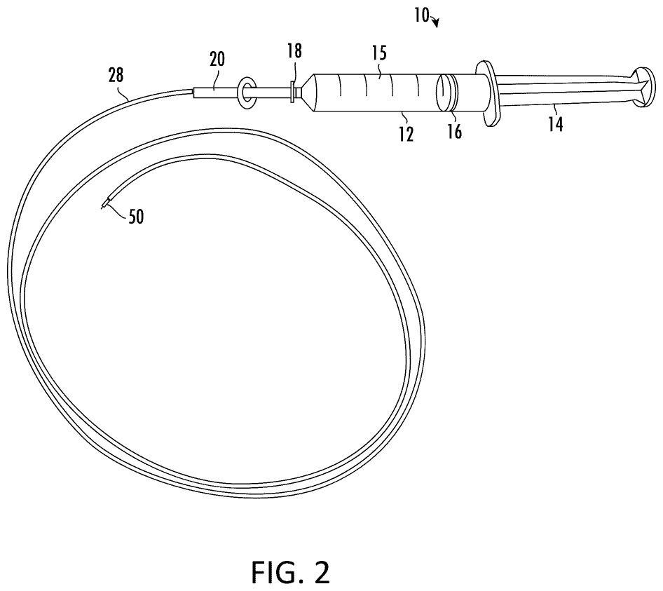

FIG. 2 illustrates a syringe 10 providing a reservoir for a radiopaque particulate suspension in accordance with the present disclosure. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a radiopaque particulate suspension 15 for injection through the needle 50.

The radiopaque particulate compositions described herein can be administered to patients for achieving a number of medical outcomes. After administration, the compositions of the present disclosure can be imaged using a suitable imaging technique as previously noted.

For example, the radiopaque particulate compositions can be injected to provide spacing between tissues, the radiopaque particulate compositions can be injected (e.g., in the form of blebs) to provide fiducial markers, the radiopaque particulate compositions can be injected for tissue augmentation or regeneration, the radiopaque particulate compositions can be injected as a filler or replacement for soft tissue, the radiopaque particulate compositions can be injected to provide mechanical support for compromised tissue, the radiopaque particulate compositions be injected as a scaffold, and/or the radiopaque particulate compositions can be injected as a carrier of therapeutic agents in the treatment of diseases and cancers and the repair and regeneration of tissue, among other uses.

The radiopaque particulate compositions of the present disclosure may be used in a variety of medical procedures, including the following, among others: a procedure to implant a fiducial marker comprising the radiopaque particles, a procedure to implant a tissue regeneration scaffold comprising the radiopaque particles, a procedure to implant a tissue support comprising the radiopaque particles, a procedure to implant a tissue bulking agent comprising the radiopaque particles, a procedure to implant a therapeutic-agent-containing depot comprising the radiopaque particles, a tissue augmentation procedure comprising implanting the radiopaque particles, a procedure to embolize tissue, including benign tumors, malignant tumors and other abnormal tissue, a procedure to introduce the radiopaque particles between a first tissue and a second tissue to space the first tissue from the second tissue.

The radiopaque particulate compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

The radiopaque particulate compositions may be injected for the permanent or temporary occlusion of blood vessels, and thus may be useful for managing various diseases and conditions. For example, the radiopaque particulate compositions may be used for the controlled, selective obliteration of the blood supply to benign and malignant tumors including treating solid tumors such as uterine fibroids, renal carcinoma, bone cancer, brain cancer, and liver cancer, among various others. The idea behind this treatment is that preferential blood flow toward a tumor will carry the embolization agent to the tumor thereby blocking the flow of blood which supplies nutrients to the tumor, causing it to shrink. Embolization may be conducted as an enhancement to chemotherapy or radiation therapy. Treatment may be enhanced in the present disclosure by including a therapeutic agent (e.g., antineoplastic/antiproliferative/anti-miotic agent, toxin, ablation agent, etc.) in the particulate composition.

Radiopaque particulate compositions in accordance with the present disclosure may also be used to treat various other diseases, conditions and disorders, including treatment of the following: arteriovenous fistulas and malformations including, for example, aneurysms such as neurovascular and aortic aneurysms, pulmonary artery pseudoaneurysms, intracerebral arteriovenous fistula, cavernous sinus dural arteriovenous fistula and arterioportal fistula, chronic venous insufficiency, varicocele, pelvic congestion syndrome, gastrointestinal bleeding, renal bleeding, urinary bleeding, varicose bleeding, uterine hemorrhage, and severe bleeding from the nose (epistaxis), as well as preoperative embolization (to reduce the amount of bleeding during a surgical procedure) and occlusion of saphenous vein side branches in a saphenous bypass graft procedure, among other uses. As elsewhere herein, treatment may be enhanced in the present disclosure by including a therapeutic agent in the particulate composition.

Radiopaque particulate compositions in accordance with the present disclosure may also be used in tissue bulking applications, for example, as augmentative materials in the treatment of urinary incontinence, vesicourethral reflux, fecal incontinence, intrinsic sphincter deficiency (ISD) or gastro-esophageal reflux disease, or as augmentative materials for aesthetic improvement. For instance, a common method for treating patients with urinary incontinence is via periurethral or transperineal injection of a bulking material. In this regard, methods of injecting bulking agents commonly require the placement of a needle at a treatment region, for example, periurethrally or transperineally. The bulking agent is injected into a plurality of locations, assisted by visual aids, causing the urethral lining to coapt. In some cases, additional applications of bulking agent may be required. Treatment may be enhanced by including a therapeutic agent (e.g., proinflammatory agents, sclerosing agents, etc.) in the particulate composition.

As previously noted, in some embodiments, radiopaque particulate composition can be injected as a bulking agent. For example, a radiopaque particulate composition in accordance with the present disclosure may be injected to increase coaptation of a bodily sphincter, such as an anal sphincter for fecal incontinence or a urinary sphincter for urinary incontinence.

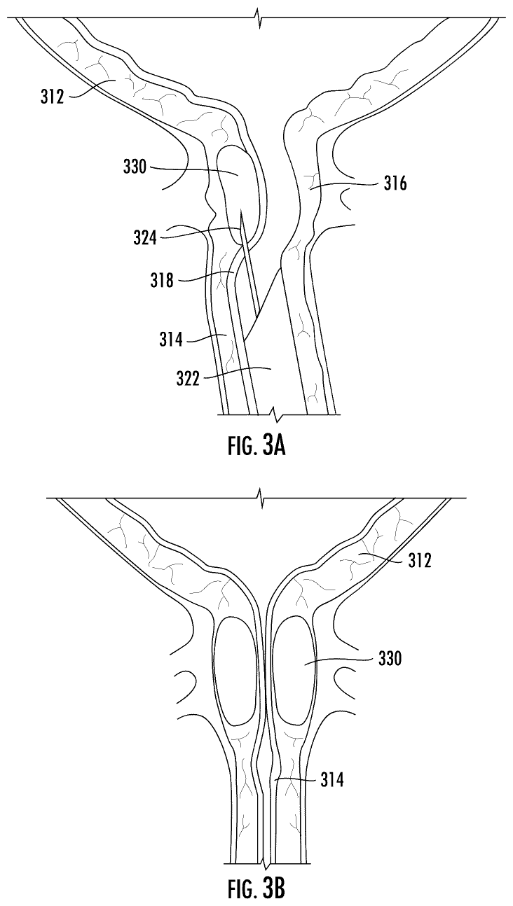

FIGS. 3A and 3B illustrate a portion of the human anatomy that includes a lower portion of the bladder wall 312, an upper portion of the urethra 314, and the bladder neck 316, which a group of muscles that connect the bladder to the urethra. A radiopaque particulate suspension in accordance with the present disclosure is injected by a transurethral route using a suitable delivery device such as that shown in FIG. 2, which includes a syringe containing the radiopaque particulate suspension (not shown in FIG. 3A), a catheter tube (not shown in FIG. 3A) and a needle 324. In the embodiment shown, the injection is performed with the aid of a cystoscope 322 through the urethral wall 318 into the bladder neck 316, increasing coadaptation of the urinary sphincter. After injection, the radiopaque particulate suspension forms a biostable implant 330.

During and/or after administration, the compositions of the present disclosure can be imaged using a suitable imaging technique, which may be, for example, an x-ray-based imaging technique, such as computerized tomography or x-ray fluoroscopy.

Although various embodiments are specifically illustrated and described herein, it will be appreciated that modifications and variations of the present disclosure are covered by the above teachings and are within the purview of any appended claims without departing from the spirit and intended scope of the present disclosure.

1. A radiopaque crosslinked acrylamide copolymer comprising (a) a residue of an iodinated monofunctional unsaturated monomer that comprises (i) a single acrylamido group, 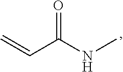
or a single methacrylate group, 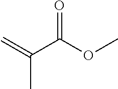
and (ii) one or more iodinated aromatic groups linked to the acrylamido group or the methacrylate group through a linkage comprising a C₂-C₈ alkyl group, (b) a residue of a monofunctional acrylamide monomer that does not contain an iodinated aromatic group, and (c) a residue of a multifunctional acrylamide monomer that does not contain an iodinated aromatic group.

2. The radiopaque crosslinked acrylamide copolymer of claim 1, wherein the linkage further comprises an amide group or an ether group.

3. The radiopaque crosslinked acrylamide copolymer of claim 1, wherein the iodinated monofunctional unsaturated monomer is selected from an iodinated (C₂-C₈ alkyl)methacrylate monomer or an iodinated N—(C₂-C₈ alkyl)acrylamide monomer, and wherein the one or more iodinated aromatic groups are linked to one or more carbons of the C₂-C₈ alkyl group of the iodinated (C₂-C₈ alkyl)methacrylate monomer or of the iodinated N—(C₂-C₈ alkyl)acrylamide monomer.

4. The radiopaque crosslinked acrylamide copolymer of claim 3, wherein the one or more iodinated aromatic groups are (a) directly linked to the one or more carbons of the C₂-C₈ alkyl group of the iodinated (C₂-C₈ alkyl)methacrylate monomer or of the iodinated N—(C₂-C₈ alkyl)acrylamide monomer, (b) linked to the one or more carbons of the C₂-C₈ alkyl group of the iodinated (C₂-C₈ alkyl)methacrylate monomer or of the iodinated N—(C₂-C₈ alkyl)acrylamide monomer through an ether group, and/or (c) linked to the one or more carbons of the C₂-C₈ alkyl group of the iodinated (C₂-C₈ alkyl)methacrylate monomer or of the iodinated N—(C₂-C₈ alkyl)acrylamide monomer through an amide group.

5. The radiopaque crosslinked acrylamide copolymer of claim 1, wherein the iodinated aromatic groups are iodinated phenyl groups.

6. The radiopaque crosslinked acrylamide copolymer of claim 1, wherein the monofunctional acrylamide monomer that does not contain an iodinated aromatic group is selected from acrylamide and an N—C₁-C₆-alkylacrylamide.

7. The radiopaque crosslinked acrylamide copolymer of claim 1, wherein the multifunctional acrylamide monomer that does not contain an iodinated aromatic group is selected from N,N′-methylenebisacrylamide, N,N′-1,2-ethylenebisacrylamide, N,N′-1,3-trimethylenebisacrylamide, N,N′-1,4-tetramethylenebisacrylamide, N,N′-1,5-pentamethylenebisacrylamide, N,N′-1,7-pentamethylenebisacrylamide, N,N′-1,6-hexamethylenebisacrylamide, N,N′-1,8-octamethylenebisacrylamide, and N,N′-1,12-dodecamethylenebisacrylamide.

8. The radiopaque crosslinked acrylamide copolymer of claim 1, wherein the radiopaque crosslinked acrylamide copolymer further comprises a residue of a multifunctional acrylamide monomer that contains one or more iodinated aromatic groups.

9. The radiopaque crosslinked acrylamide copolymer of claim 8, wherein the multifunctional acrylamide monomer that contains one or more iodinated aromatic groups is selected from 1,3-bisacrylamido-2,4,6-triiodobenzene, 1,3-bisacrylamido-5-iodobenzene, and 1,3-bisacrylamido-2,4,5,6-tetraiodobenzene.

10. The radiopaque crosslinked acrylamide copolymer of claim 1, wherein the radiopaque crosslinked acrylamide copolymer is a hydrogel.

11. The radiopaque crosslinked acrylamide copolymer of claim 1, wherein the radiopaque crosslinked acrylamide copolymer has a radiopacity that is greater than 100 Hounsfield units (HU).

12. A radiopaque crosslinked acrylamide copolymer comprising (a) a residue of an iodinated monofunctional unsaturated monomer that comprises (i) a single acrylamido group, 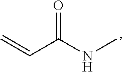
or a single methacrylate group, 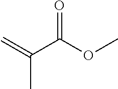
and (ii) one or more iodinated aromatic groups, (b) a residue of a monofunctional acrylamide monomer that does not contain an iodinated aromatic group, and (c) a residue of a multifunctional acrylamide monomer that does not contain an iodinated aromatic group, wherein the one or more iodinated aromatic groups in the radiopaque crosslinked acrylamide copolymer are spaced from a polymer chain into which the residue of the iodinated monofunctional unsaturated monomer is incorporated through a linkage that comprises an alkyl chain that ranges from two to eight carbon atoms in length.

13. A radiopaque particulate composition comprising radiopaque particles that comprise a radiopaque crosslinked acrylamide copolymer comprising (a) a residue of an iodinated monofunctional unsaturated monomer that comprises (i) a single acrylamido group, 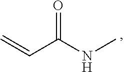
or a single methacrylate group, 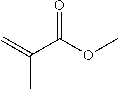
and (ii) one or more iodinated aromatic groups linked to the acrylamido group or the methacrylate group through a linkage comprising a C₂-C₈ alkyl group, (b) a residue of a monofunctional acrylamide monomer that does not contain an iodinated aromatic group, and (c) a residue of a multifunctional acrylamide monomer that does not contain an iodinated aromatic group.

14. The radiopaque particulate composition of claim 13, further comprising one or more additional agents selected from one or more of therapeutic agents, imaging agents, colorants, tonicity adjusting agents, suspension agents, wetting agents, and pH adjusting agents.

15. The radiopaque particulate composition of claim 13, wherein the radiopaque particles have an average size ranging from 50 to 950 microns.

16. The radiopaque particulate composition of claim 13, wherein the radiopaque particulate composition is an injectable composition.

17. The radiopaque particulate composition of claim 13, wherein the radiopaque particulate composition is provided in a container in sterile dry form or wherein the radiopaque particulate composition is provided in a container in a suspension.

18. The radiopaque particulate composition of claim 17, wherein the container is selected from a syringe, vial, ampoule or catheter component.

19. A medical procedure comprising administering to a subject a radiopaque particulate composition in accordance with claim 13.

20. The medical procedure of claim 19, wherein the medical procedure is selected from a tissue bulking procedure, a tissue spacing procedure, a blood vessel occlusion procedure, and a fiducial marking procedure.
